(19) United States
(12) Patent Application Publication
(10) Pub. No.: US 2024/0374765 A1
(43) Pub. Date: Nov. 14, 2024

(54) PLASMA SOURCE FOR HAND DISINFECTION

(71) Applicant: DBD PLASMA GMBH, Göttingen (DE)

(72) Inventors: Roland DAMM, Ebergötzen (DE); Alexander GREDNER, Friedland (DE); Christian HAESE, Gleichen (DE); Jürgen HAESE, Gleichen (DE)

(73) Assignee: DBD PLASMA GMBH, Göttingen (DE)

(21) Appl. No.: 18/564,609

(22) PCT Filed: Mar. 22, 2022
(86) PCT No.: PCT/EP2022/057487

§ 371 Date: Nov. 27, 2023

(30) Foreign Application Priority Data
May 28, 2021 (WO) PCT/EP2021/064350

(51) Int. Cl. A61L 2/00; A47K 10/48; A61L 2/26; H05H 1/24

(52) U.S. Cl. CPC A61L 2/0029; A47K 10/48; A61L 2/26; H05H 1/2439; A61L 2202/11; A61L 2202/122; A61L 2202/15; H05H 2245/34

(57) ABSTRACT

An apparatus that dries and disinfects areas of the body without contact. A disinfecting effect is produced by a plasma generator which imparts the disinfecting effect to an air stream heated for hand drying. The plasma generator generates reactive plasma species, which are brought to human skin by the air stream. Contact with the plasma source and high-voltage components is avoided. The invention also relates a method for disinfecting human skin.

### CROSS-REFERENCE TO RELATED APPLICATIONS

This application is a United States National Stage Application of International Application No. PCT/EP2022/057487 filed Mar. 22, 2022, claiming priority from International Patent Application No. PCT/EP2021/064350 filed May 28, 2021.

### FIELD OF THE INVENTION

The invention preferably relates to an apparatus that dries and disinfects areas of the body, in particular hands, without contact. The disinfecting effect is produced by a plasma generator, which imparts the disinfecting effect to an air stream for hand drying. The plasma generator produces reactive plasma species, which are brought to the human skin by the air flow. Contact with the plasma source and high-voltage components is avoided.

Furthermore, the invention relates to a use of the apparatus according to the invention and a method for disinfecting human skin.

### BACKGROUND OF THE INVENTION

Personal hygiene, in particular hand hygiene, has become a matter of course these days. Washing hands serves to remove dirt and potential pathogens. It therefore has both an aesthetic and a hygienic function. After washing the hands, they are dried, whereby in addition to textile or paper towels, automatic hand dryers are also known in the prior art.

Hand dryers generally generate an air flow that is intended to reduce and dry the moisture on the hands after washing. With conventional hand dryers, the focus is on drying. In most cases, hand dryers do not have an additional hygienic function for disinfection.

In addition to acting as a dryer, hand dryers and/or air purifiers with plasma sources, ion sources or UV lamps are also known in the prior art to disinfect flowing air, body areas or entire room areas. Ions, in particular reactive species, are generated with the aid of such sources. The reactive species react with the cells of potential pathogens on the skin such that these are eliminated, for example through membrane and/or DNA damage. Hand dryers with this type of mode of action therefore offer a promising alternative to conventional hand dryers that are only intended for drying.

Some of these apparatuses known in the prior art are briefly described below.

US 2004/0184972 A1 discloses a method and an apparatus for purifying room air. The apparatus comprises a plasma generator or an atmospheric plasma device and a filter medium. The filter medium can receive and filter the entire air flow. The atmospheric plasma is generated by either a radio frequency (RF) electric field, an alternating current (AC) electric field or a direct current (DC) field. The apparatus and the process are not intended for disinfecting hands or other areas of the body.

US 2007/0253860 A1 discloses an apparatus and the use of a device which purifies room air. In a first step, the air is irradiated with a UV radiation source in order to kill microorganisms. In a second step, the air is passed through a catalyst in order to break down the ozone produced by the UV radiation. In a third step, the air is ionized.

US 2013/0233172 A1 describes a room air purifier that works with an electrostatic precipitator. The basic principle of this invention is corona discharge. The corona discharge is formed by applying a high voltage to a thin wire. The charge carriers released as a result charge particles carried along with the air flow, which then deposit on a metal plate.

DE 10 2008 063 052 A1 describes an apparatus for purifying room air, in particular for preventing odors. Its housing contains a device for generating plasma such that germs or unpleasant odors can be eliminated.

WO 2015/032888 A1 describes a disinfection apparatus and a method for plasma disinfection of surfaces with a plasma generator for generating a disinfecting plasma gas stream. The apparatus comprises an aerosol generator for generating an aerosol stream containing aqueous particles, wherein the aerosol generator with the plasma generator directs a mixed plasma gas stream in the disinfection area onto the surface to be disinfected.

EP 1925190 B1 discloses a plasma source, in particular for disinfecting wounds, which comprises an ionization chamber with an inlet for introducing a gas into the ionization chamber and a plurality of ionization electrodes located inside the ionization chamber. The ionization electrodes ionize the gas inside the ionization chamber by emitting microwaves.

DE 102008054401 discloses a dryer in which a cavity accessible from the outside through a housing opening is designed to accommodate areas of the body, in particular the hands, to be dried by means of an air flow in the cavity. A plasma or ion source, preferably a microwave or high-frequency source, is used to reduce germs in the air flow.

WO 2015/132368 A1 discloses a disinfection apparatus which has an electrode arrangement which is arranged transversely to an air flow path. The electrode arrangement causes a plasma discharge along its longitudinal axis, such that the air flowing through it is exposed to the plasma. The electrode arrangement consists of a grid. The plasma only forms on the surface of the dielectric, on which a first grid structure is located. However, the air flow is slowed down considerably by the close-meshed grid, resulting in a thick flowing boundary layer. This prevents good mixing of the reactive species of the plasma, which are used for disinfection, with the surrounding air. They may even be partially broken down again in the grids. In addition, due to the structure of the grid, the required power per surface is only determined by the frequency and voltage. A reduction in surface power is only possible to a limited extent by reducing the voltage, as otherwise no stable plasma can be generated. In addition, small turbulences generated by the plasma itself are intercepted by the narrow meshes of the grid and cannot have any effect in terms of disinfection.

In light of the prior art, there is therefore a need for an improved system for more efficient use of plasma sources for hand drying, hand disinfection and/or room air disinfection.

### OBJECT OF THE INVENTION

The objective of the present invention was to eliminate the disadvantages of the prior art and to provide an improved apparatus for disinfecting body areas on the basis of a plasma source in an air stream. In particular, an apparatus for disinfecting body areas should be provided which is characterized by a particularly effective generation and transport of reactive plasma species with a disinfecting effect to the desired disinfection area.

### SUMMARY OF THE INVENTION

The objective according to the invention is solved by the features of the independent claims. Advantageous embodiments of the invention are described in the dependent claims.

In a preferred embodiment, the invention relates to an apparatus for disinfecting body areas comprising a fan for generating an air flow and a plasma generator, wherein the plasma generator is located in the air flow, characterized in that the plasma generator comprises at least one plasma rod, which comprises a dielectric tube having an electrically conductive core within the dielectric tube, the dielectric tube having on an outer side a wire coiled into windings, the electrically conductive core forming with the wire coiled around the outer side a pair of electrodes which generates a plasma when a voltage is applied.

Preferably, the apparatus according to the invention is designed to dry and disinfect areas of the body, in particular hands, after washing. For this purpose, the fan causes an air flow, which preferably flows transversely to the plasma generator. As the air flow passes the plasma generator, a so-called cold plasma is preferably created by the principle of dielectric barrier discharge. When this cold plasma is generated, reactive oxygen and nitrogen species are preferably formed, which are carried along by the flowing air stream and hit the wet areas of the body, especially the wet hands, after the washing process. Without wishing to be bound by any particular theory, the advantageous disinfecting effect of the apparatus according to the invention can preferably be attributed at least in part to the provision of such reactive oxygen and nitrogen species. The reactive oxygen and nitrogen species encounter potential pathogens in wet areas of the body. In particular, the reactive oxygen and nitrogen species, especially the singlet oxygen ¹O₂ react advantageously with the carbon double bonds of the amino acids of spores, fungi, germs, bacteria and/or viruses. These potential pathogens are killed by DNA and/or membrane damage.

The apparatus according to the invention therefore preferably causes the moisture on areas of the body, for example on the hands after washing them, to have a disinfecting effect. It is particularly advantageous that the apparatus according to the invention acts simultaneously as a dryer and a disinfectant. Commercially available dryers, in particular hand dryers, which are located for example in the toilets of businesses, restaurants, etc., usually only fulfill the function of drying. To disinfect hands, commercially available disinfectants in liquid containers are often placed at the sink. A person can use these after washing their hands. Such disinfectants consist of chemical compounds that eliminate potential pathogens, but can also have a negative effect on the underlying skin, for example through allergic reactions. The apparatus according to the invention avoids this advantageously, as no disinfectants are required. Instead, the moisture that is present on the body areas, for example the hands, after washing preferably has a disinfecting effect. This is preferably achieved by dissolving the reactive oxygen and nitrogen species in the liquid, which is located on the hands, for example, whereby potential pathogens can be eliminated.

The physical principle of dielectric barrier discharge, which is preferably used in the apparatus according to the invention and which preferably leads to the formation of the plasma, will be explained in more detail below.

A dielectric barrier discharge is preferably generated by applying a high voltage between two electrodes, wherein at least one of the electrodes is insulated by a dielectric. The use of insulation prevents the formation of an arc discharge. Instead, many fine plasma filaments preferably form between the electrodes, but these only have a short life in the range of a few nanoseconds. The reason for this is the accumulation of charge carriers on the surface of the dielectric, which generate an opposing field to the externally applied voltage, such that the discharge is extinguished again. For this reason, an alternating voltage of typically a few kilohertz is used.

The dielectric barrier discharge can be generated both at low pressure and at atmospheric pressure, wherein its special properties are primarily used at atmospheric pressure. Due to the large mass of the ions compared to the electrodes, the heavy ions can absorb far less energy from the alternating field than the lighter and faster electrons. It is therefore preferably a non-thermal “cold” plasma, in which the electrons have a high temperature, but the neutral gas and the ions are at around room temperature. This means that temperature-sensitive materials, such as human skin and/or the surrounding air, can also be treated with this type of discharge.

Dielectric barrier discharge is used for a wide range of applications, including ozone production, as a UV source, for air and wastewater treatment, for sterilization, as well as for activating, cleaning, etching and coating surfaces. A major advantage of dielectric barrier discharge is that it can be produced in different shapes and sizes through flexible electrode and dielectric arrangements and can therefore be easily adapted to a specific application.

In the context of the invention, dielectric barrier discharge is preferably used to disinfect areas of the body and/or the surrounding air.

Numerous reactive species are formed in the plasma. However, only a few of these reactive species are so long-lasting that they continue to have an effect outside the discharge area. The reactive oxygen and nitrogen species are an exception. These are either stable, such as the singlet oxygen 102, and interact directly with the target molecules or radicals are formed, which are also long-lasting. For example, the superoxide radical (O₂·⁻) is formed in the plasma, which reacts with nitric oxide (NO·) to form reactive nitrogen species comprising the peroxinitrites (ONOO⁻) and (ONOOH). Without wishing to be bound to a particular theory of the mode of action, the advantageous disinfecting effect of the apparatus according to the invention can preferably be attributed at least in part to the provision of the aforementioned reactive species.

When disinfecting areas of the body, especially the hands, the reactive oxygen and nitrogen species are preferably transported to the affected areas. This occurs preferably by means of an air flow. The air flow is preferably generated by a fan. The term “fan” can also be used synonymously with the term “ventilator”.

For the purposes of the invention, an air flow preferably refers to the movement of air. Air behaves fluidically, i.e. particles naturally flow from areas with higher pressure to areas where the pressure is lower.

For the purposes of the invention, a fan preferably refers to a driven turbomachine that conveys a gaseous medium, preferably air. For this purpose, the fan can have an impeller with axial or radial flow, which preferably rotates in a housing of the apparatus according to the invention. For example, a pressure ratio of between 1 and 1.3 can be achieved between the intake and discharge sides. Turbomachines that achieve a pressure ratio greater than 1.3 are usually referred to as compressors. Instead, fans preferably convert a maximum of 25 KJ/(kg×K), which corresponds to 30,000 Pa (Pascal) at an assumed density of 1.2 kg/m³ (air), hence the factor 1.3 according to DIN 5801 and 13349.

Various designs of fans are known in the prior art which can be used in the apparatus according to the invention.

Axial fans are very common designs. The axis of rotation of the axial impeller runs parallel (axially) to the air flow. The air is moved by the axial impeller in a similar way to an aircraft or ship propeller. Axial fans achieve a high throughput with small dimensions. The achievable pressure ratio is lower than with radial fans. The pressure build-up is caused by the incoming air being deflected by the blades of the impeller and leaving the fan on spiral paths. The pressure build-up depends on the angle that the air flow forms relative to the blade profile. If more pressure is to be achieved, this angle must be increased. However, if the angle of incidence is too large, the profile flow breaks off and the fan operates inefficiently and with more noise.

Radial fans are used wherever a greater increase in pressure is required for the same air volume compared to axial fans. The air is drawn in axially (parallel to the drive axis) of the radial fan and deflected by 90° by the rotation of the radial impeller and blown out radially. A distinction is made between impellers with backward-curved blades (for high pressures and efficiencies), straight blades (for special purposes such as particle-laden flows to reduce build-up) and forward-curved blades (for low pressures and efficiencies). There are single-sided and double-sided intake radial fans with and without housing. The air is usually blown out at a flange or a pipe socket. In order to minimize pressure losses due to the high outlet velocity from the radial fan, care must be taken to ensure a suitable design of the continuing duct (with a diffuser if necessary).

So-called diagonal fans are also known. Here, the air is not discharged axially, but diagonally. Diagonal fans have a higher air flow rate and build up a higher pressure with the same power and size, which is why they can be operated at a lower speed with the same effect and are therefore quieter.

Tangential fans, which have blades pointing in the direction of rotation, are also known. With tangential fans, the air is guided through the fan wheel twice. It is drawn in tangentially over a large area, approximately half the surface of the fan wheel, passed through the inside of the wheel and also discharged tangentially. The wheel also transports a small amount of air on the outside. The air then usually exits via a narrow gap the width of the fan wheel on the opposite side. Compressors that generate compressed air and ionic winds generated by additional DC voltage sources are also known.

Advantageously, all of the aforementioned variants of the fans can preferably be used in an apparatus according to the invention.

The ions and/or reactive oxygen and nitrogen species, which are carried by the air flow to the surrounding air or to the areas of the body to be disinfected, are generated by the plasma generator.

For the purposes of the invention, a plasma generator is a component of the apparatus according to the invention which generates a plasma and thus preferably the reactive species.

In particular, a plasma generator comprises at least one dielectric tube with a conductive core, wherein the dielectric tube exhibits a wire coiled around an outer side.

For the purposes of the invention, the dielectric tube may also be referred to as a dielectric conductive tube. The dielectric tube preferably comprises a dielectric material. For the purposes of the invention, a dielectric material preferably refers to a material which does not conduct electrical current or only conducts it very weakly.

The dielectric tube exhibits a wire on its outside. This wire is coiled into windings along this outer side. The electrically conductive core inside and the wire coiled into windings outside the dielectric tube form a pair of electrodes. The dielectric tube with the pair of electrodes is also referred to as a plasma rod for the purposes of the invention. The application of a voltage, preferably an alternating voltage, preferably produces the dielectric barrier discharge and thus the plasma and thus preferably the reactive species along the surface of the dielectric tube.

Preferably, the wire on the outside of the dielectric tube is coiled twice and in opposite directions. This compensates for the inductance that this coiling could have. The double coiling achieves a structure similar to that of a coaxial cable, which does not emit any electromagnetic radiation. Instead, the current-carrying coils can effectively serve to generate the plasma. The air flow around the dielectric tube is preferably in a transverse direction due to the fan.

If an attempt is made to distribute a dielectric barrier discharge over a large effective area, thermal instability must be taken into account. The reason for this is that plasma ignites more easily where the electrode or dielectric is warmer. If the applied voltage is only slightly higher than the voltage required for ignition, the plasma will usually only ignite at the points where the temperature is higher. At other points, it may not ignite at all. This can be effectively remedied by increasing the voltage, which increases the power, or by reducing the frequency, which in turn requires an increase in the size of any transformers present.

In the apparatus according to the invention, the problem is solved by the fact that the plasma preferably only ignites along the thin line of the wire coil. The wire can be coiled with almost any distance between the windings. This allows the power to be reduced to a desired level despite the high frequency and high voltage without reducing the length of the tube. This means that the generation of reactive species can be distributed over a greater distance or over a larger area and thus a larger flow profile can be evenly supplied with reactive species.

Preferably, the dielectric tube exhibits a smooth surface. Preferably, a smooth surface refers to a surface having an average roughness value between 0 μm and 500 μm, preferably 0 and 50 μm or particularly preferably up to 5 μm, up to 1 μm. A person skilled in the art knows that the average roughness value is a parameter for describing the roughness or smoothness, whereby the average roughness value (Rₐ) indicates the arithmetic mean of the amount of deviation from a center line of the surface and is generally given in μm (micrometers) as a unit.

Advantageously, the smooth surface of the dielectric tube causes the air to flow smoothly around it, whereby the boundary layer in which the air adheres to the surface of the dielectric tube is relatively thin. This is advantageous in that the air hardly flows in this boundary layer. The reactive species must therefore diffuse through this layer. Furthermore, this boundary layer is disturbed at a few points by the coiled wire. This causes turbulence, which is advantageous as the mixing of the reactive species with the air flowing around them is considerably improved.

In contrast to WO 2015/132368 A1, the air flow is not strongly decelerated, as the coiled wire is preferably not as close-meshed as the grid. Instead, the boundary layer of the apparatus according to the invention is preferably significantly thinner, such that the reactive species are mixed particularly well with the air flowing around them. In addition, the thin boundary layer means that reactive species in the apparatus according to the invention are not broken down again after generation.

It is also known from the prior art that discharge processes themselves can have an influence on flows, in particular on the air flowing around them. One reason for this can be acoustic emissions due to the sudden heating of the air. Another reason may be the ionic wind. In ionic wind, the ions generated by the discharge on the thin wire are attracted to the surface of the dielectric tube and drag other gas particles with them. These effects are usually weak. However, they cause a flow along the surface of the dielectric tube, which is stronger the greater the plasma output per wire length.

In a preferred embodiment, the apparatus is characterized in that the plasma generator comprises an array of a plurality of plasma rods.

For the purposes of the invention, an array refers to an arrangement structure of the plasma generator or the plasma rods. Preferably, several plasma rods forming an array can represent the plasma generator.

Preferably, the array is one-, two- and/or three-dimensional. The plasma rods are preferably interconnected in one, two and/or three dimensions by parallel circuits. A parallel connection is advantageous because if one plasma rod fails, the other plasma rods of the plasma generator retain their functionality.

In a further embodiment, a series connection of two or more plasma rods may also be preferred. In the case of a symmetrical power supply, the two or more rods can be operated symmetrically, which further reduces electromagnetic emissions.

The design as an array results in a compact construction of the plasma generator, such that a large number of plasma rods can be installed.

Another advantage of the plasma generator's array design is its easy and simple scalability. This means that the plasma generator can be easily and efficiently adapted to planned operating and load conditions.

Furthermore, the design as an array is advantageous in that the dielectric barrier discharge is applied in strips, areas and/or volumes so that the reactive species can be formed over a large area. The air flow can therefore be used to dry and simultaneously disinfect larger areas of the body.

In a further preferred embodiment, the apparatus is characterized in that the dielectric tube has a wall thickness of between 0.5 mm and 3 mm, preferably between 1 mm and 2 mm.

For the purposes of the invention, the wall thickness preferably refers to the difference between the inner and outer dimensions of the dielectric tube. The measurement is preferably made perpendicular to the wall extension or perpendicular to the body axis. The inner and outer diameters are required to calculate the wall thickness of the dielectric tube. The wall thickness is an important parameter for describing the functionality of components, particularly with regard to their statics, dynamics and strength.

The specified wall thicknesses between 0.5 mm and 3 mm, preferably between 1 mm and 2 mm, have proven to be particularly advantageous, especially with regard to stability and dielectric strength.

In addition, the preferred wall thicknesses advantageously increase the dielectric strength and prevent voltage breakdowns.

The dielectric strength of the dielectric tube is preferably the maximum electric field strength E that may prevail in the dielectric tube without a voltage breakdown occurring. Accordingly, it is also referred to as the breakdown field strength. It is calculated from the breakdown voltage U in relation to the wall thickness d of the dielectric tube E=U/d.

During the voltage breakdown, a channel is formed for a certain period of time in which an electrically conductive plasma is created from the material of the dielectric tube through heat and ionization. The radiation emitted by the plasma, for example ultraviolet radiation, preferably knocks further electrons out of the material of the dielectric tube, which further increases the conductivity in the channel. Depending on the power source, the breakdown can quickly extinguish as a spark or continue to burn as an arc. The insulating material, in this case the dielectric tube, is often irreversibly changed or even destroyed along the path taken by the spark. Plastics can be partially carbonized by the heat of the spark and are then unusable as insulators. It is therefore important to avoid voltage breakdowns. Advantageously, the specified wall thicknesses are particularly effective in preventing such voltage breakdowns.

In a further preferred embodiment, the apparatus is characterized in that the dielectric tube has a length of between 4 and 30 times the outer diameter of the dielectric tube.

The preferred lengths have proven to be particularly advantageous, as they provide a sufficient area for generating the plasma and at the same time ensure a high degree of stability of the dielectric tube.

The dielectric tube preferably comprises typical materials that are suitable for dielectrics in high-frequency applications. Preferred materials for the dielectric tube include polyethylene, polytetrafluoroethylene (PTFE), ceramic, for example steatite and/or aluminum oxide, mica and/or glass, for example borosilicate glass. A person skilled in the art knows that a material should be selected for the dielectric tube which preferably also has low dielectric loss factors.

In a further preferred embodiment, the apparatus is characterized in that the wire is coiled into windings in opposite directions over at least twice the length of the tube and a plasma distributed over the length of the tube is generated as a function of the applied voltage.

For the purposes of the invention, a winding refers to a coil of the wire along the surface of the dielectric tube around the longitudinal axis of the dielectric tube. Therefore, a winding in the sense of the invention can also be referred to as a coil. Preferably, the resulting geometric structure resembles a screw or spiral shape. Preferred are a plurality of windings along a length of the tube, wherein one winding of the wire preferably corresponds to one rotation of the dielectric tube and a plurality of windings preferably correspond to a plurality of successive rotations of the dielectric tube along a coiling direction.

Coiling the wire in opposite directions along twice the length of the dielectric tube advantageously results in so-called meshes, which are each formed by coiling the wire in opposite directions. A counter-rotating coiling of the wire refers to the wrapping of the wire along the dielectric tube, with at least one winding in the forward direction and one winding in the reverse direction.

The wire can preferably be attached to the dielectric tube manually, by etching and/or gluing. Automated coiling can also be used. All processes known in the prior art can be used for this purpose.

The plasma is preferably formed along the thin line of the wire coil by applying a high voltage. Therefore, the wire can advantageously be coiled with almost any distance between the turns. This means that the output can be reduced to a desired level despite the high frequency and high voltage without reducing the length of the dielectric tube. During the formation of the plasma, reactive species are preferably produced, which are thus distributed over a greater distance, in particular over the length of the dielectric tube, and over a larger area. Therefore, a larger flow profile can advantageously be evenly supplied with reactive species.

In a further preferred embodiment, the apparatus is characterized in that the windings of the wire are spaced apart by a distance of up to 10 times the outer diameter of the dielectric tube, preferably between 1 and 10 times the outer diameter, more preferably between 1 and 5 times the outer diameter, particularly preferably between 2 and 4 times the outer diameter. The distance between the windings preferably means the average distance between windings of the wire in a coiling direction. Preferably, the distance between windings of the wire can be measured along the longitudinal axis on an outer contour of the wire. In the case of a coiling of the wire with four windings in one coiling direction over a length of 80 mm, for example, the distance is approx. 20 mm.

It has proven to be particularly advantageous to space the windings such that the circulating air preferably creates a relatively thin boundary layer along the dielectric tube. The thin boundary layer allows the reactive species produced during plasma formation to mix particularly well with the circulating air and transport them to areas of the body to be dried and/or disinfected. A thick boundary layer is avoided by the specified distances between the windings of the wire, which in turn would adversely affect particularly good mixing of the reactive species.

Terms such as substantially, approximately, about, approx. etc. preferably describe a tolerance range of less than ±20%, preferably less than ±10%, even more preferably less than ±5% and in particular less than ±1%. Terms such as substantially, approximately, about, approx. etc. always disclose and include the exact stated value.

The air, in particular the reactive species formed by the plasma, is preferably exchanged along the coil of the wire by the air flow through the apparatus according to the invention. In particular, the reactive species can easily reach the surrounding air outside the apparatus according to the invention through the circulating air, which preferably passes through the dielectric tube in the transverse direction.

These advantageous effects represent significant differences to the prior art. For example, due to the grid structure in WO 2015/132368 A1, an exchange of air and thus reactive species in the narrow grid structures is possible to a lesser extent, such that the reactive species can even be degraded in the plasma itself and thus do not reach the body areas to be dried and/or disinfected and/or the ambient air. This is advantageously avoided by the wire coil, in particular by the preferred spacing of the windings of the wire.

The wire for coiling around the dielectric tube can comprise materials that are known in the prior art as good conductor materials. These can preferably be materials such as iron, steel, brass, aluminum, gold, titanium, copper and/or copper alloys. Preferred materials are those that are electrically conductive and have a high resistance to corrosion. The term corrosion is defined in DIN EN ISO 8044. A person skilled in the art is able to select suitable materials for the wire.

In a further preferred embodiment, the apparatus is characterized in that the wire has a diameter of between 0.1 mm and 1 mm, preferably between 0.2 mm and 0.4 mm.

Preferably, the dielectric tube has a smooth surface. This is advantageous, as the smooth surface of the dielectric tube is partly responsible for a smooth flow of air around it, whereby the boundary layer along the dielectric tube is relatively thin, with the boundary layer itself hardly flowing at all. The above-mentioned dimensions with regard to the diameter of the wire are particularly advantageous for the formation of a thin boundary layer. The fact that the wire has such small dimensions means that the smooth surface of the dielectric tube is maintained, which is advantageous for the air flow around it, as the air itself flows smoothly around the dielectric tube. Furthermore, the thin boundary layer is preferably slightly disturbed by the thin wire. This leads to minimal turbulence, which is advantageous, however, as the reactive species are mixed much better with the air flowing around them.

The plasma or dielectric barrier discharge created by the apparatus according to the invention preferably itself exerts an influence on the air flowing around it. These are on the one hand acoustic emissions, which are caused by the heating of the air, and on the other hand an ion wind, in which ions created on the wire are attracted to the surface of the dielectric tube and entrain other gas particles. Although these effects are relatively weak, they cause a flow along the surface of the dielectric tube, which is all the stronger the greater the output of the plasma per length of wire.

In a further preferred embodiment, the apparatus is characterized in that the electrically conductive core comprises a wire. The electrically conductive core can be a solid wire or a tube, the dimensions of which are preferably such that it fits into the dielectric tube. For example, the electrically conductive core can be in the form of a tube which consists of a wire mesh and whose dimensions are such that the electrically conductive core fits into the dielectric tube.

The electrically conductive core and the wire coiled along the dielectric tube form a pair of electrodes. The dielectric tube preferably shields the conductive core from the air flowing around it. An electric field also forms between electrically insulated electrodes. If the field strength is high enough, an electrical discharge occurs. However, the insulation preferably does not result in a current flow, which is why the gas does not heat up. This is where the term “cold plasma” comes from. The electrons migrate to the next electrode and preferably cannot flow away due to the insulation. The resulting charge preferably compensates for the applied high-voltage field after a very short time and extinguishes the discharge. In order to maintain the gas discharge, it is therefore preferable to carry out the excitation with a high-frequency alternating voltage, which is applied to both electrodes. In itself, the transport of ions is preferably largely suppressed by the described principle of dielectric barrier discharge, but they can be transported by the supplied air flow, which preferably results from the fan, and in particular used for disinfection.

The principle of dielectric barrier discharge has further advantages. In particular, dielectric barrier discharge is highly efficient, as no charge carriers have to enter or leave the electrodes. This eliminates the cathode case so that no glow emission is necessary. In particular, it is an advantage that the effect of the dielectric barrier discharge can take place in normal atmospheric air, especially in the air flowing around it.

In a further preferred embodiment, the apparatus is characterized in that the apparatus comprises a voltage source and/or a transformer for providing a desired voltage, preferably an alternating voltage.

A transformer preferably comprises two or more coils, which are usually coiled from insulated copper wire and are located on a common magnetic core. The transformer converts an AC input voltage that is applied to one of the coils into an AC output voltage that can be tapped at the other coil.

It is known that the coil is referred to as the primary coil, to which an alternating voltage U_{P} is applied from the outside, and the coil at which the output voltage U_{S} is tapped is referred to as the secondary coil. According to the law of induction, an alternating voltage on the primary coil of the transformer causes a changing magnetic flux in the core. The changing magnetic flux in turn induces a voltage on the secondary coil of the transformer, which is also referred to as voltage transformation. According to Ampère's law, an alternating current in the secondary coil causes an alternating current in the primary coil, which is also known as current transformation. The voltage ratio between the primary and secondary coils is given by the ratio of their number of windings according to the transformer equation. By selecting the appropriate number of windings, a transformer can therefore be used to transform AC voltages both upwards and downwards.

While a direct voltage always has the same polarity and therefore the same direction of flow for a direct current, a voltage whose polarity changes periodically is preferably referred to as an alternating voltage. Accordingly, the direction of flow of the alternating current preferably changes periodically. Voltage and current do not necessarily have to have the time course of a sinusoidal function. However, sinusoidal alternating current is technically the most widespread, as it is generated during electricity generation in alternating current generators.

For alternating voltages and alternating currents, the effective values for voltage and current are usually specified. They differ from the average values and the maximum values.

The effective value of an alternating voltage is preferably understood to be the constant voltage over time (direct voltage) that delivers the same energy as the alternating voltage at the same resistance R in the same time.

The effective value of an alternating current is preferably understood to be the constant current intensity (current intensity of a direct current) that delivers the same energy as the alternating current at the same resistance R in the same time.

It may also be preferable for a direct voltage to be applied and for this to be converted into an alternating voltage by an inverter. A person skilled in the art knows ways of providing an alternating voltage, which is the preferred means of supplying the apparatus according to the invention.

In a further preferred embodiment, the apparatus is characterized in that the applied voltage is an alternating voltage, preferably with an effective value between 1 kV (kilovolt) and 10 kV and/or a frequency between 1 kHz (kilohertz) and 500 kHz.

In a further preferred embodiment, the apparatus is characterized in that the applied voltage is an alternating voltage, preferably with a peak-to-peak value between 3 kV (kilovolts) and 15 kV and/or a frequency between 1 kHz (kilohertz) and 500 kHz.

For example, a sinusoidal and/or rectangular voltage curve can be selected for the alternating voltage.

A sinusoidal voltage curve is also referred to as a sinusoidal voltage. Mathematically, it is described by the product of the peak value of the alternating voltage and the sine function, whereby the product of angular frequency and time is formed as the argument of the sine function, whereby this product is also referred to as the phase angle.

A rectangular voltage curve preferably refers to a periodic signal that switches back and forth between two values and has a rectangular curve in a diagram over time.

It may also be preferable to use other signal curves, for example a triangular, sawtooth or trapezoidal voltage curve. It may also be preferable to use high-voltage pulses with different amplitudes or repetition rates in order to provide a decaying alternating voltage. Preferred voltage pulses can, for example, have the form of a decaying sine wave. In this case, the voltage can preferably be essentially composed of a positive and a similarly sized negative half-wave, followed by a plurality of post-oscillations. For the frequency of a decaying alternating voltage, for example, between 100 kHz (kilohertz) and 1000 kHz, preferably 500 kHz to 1000 kHz can be used, whereby a repetition rate of the voltage pulses can be selected, for example, from a range of 100 Hz to 10 KHz.

The choice of signal curves for the preferred alternating voltage can be handled by a person skilled in the art, who is able to decide which one to use depending on the application.

In a further preferred embodiment, the apparatus is characterized in that the plasma comprises a mixture of particles at the atomic-molecular level, preferably reactive oxygen and nitrogen species.

For the purposes of the invention, a plasma preferably refers to a mixture of particles comprising ions, free electrons and usually also atoms and/or molecules. A plasma therefore contains free charge carriers. The degree of ionization of a plasma can be less than 1%, but can also be 100% (complete ionization). A substantial property of plasmas is their electrical conductivity.

In the case of dielectric barrier discharge, the plasma, preferably the cold plasma already mentioned, is non-thermal, i.e. the plasma is not in thermal equilibrium. In concrete terms, this means that the temperatures of the contained particle types differ significantly.

In the case of dielectric barrier discharge, a plasma is generated far from thermal equilibrium despite normal air pressure. The high-frequency alternating voltage causes tiny discharge channels to form preferentially with each period. These discharge channels are also referred to as “streamers” in the prior art. As the discharge channels only make up a fraction of the total discharge volume and the duration of the discharge is severely limited by the capacitive coupling, the average gas temperature in the discharge remains low and therefore close to room temperature.

In particular, the plasma contains numerous reactive species, preferably reactive oxygen and nitrogen species. Reactive species are characterized by the fact that they are highly reactive and can have a disinfecting effect in the context of the invention. The reactive oxygen and nitrogen species are therefore preferred for the apparatus according to the invention, since they are so long-lasting that they can also act outside the discharge area. In particular, they are stable over a period of time such that they can be carried by the flowing air around the body areas to be disinfected, in particular the hands, and/or the ambient air.

Reactive oxygen species (ROS) are also referred to simply as “oxygen radicals”. ROS are highly reactive oxygen-containing molecules. Reactive nitrogen species (RNS) are highly reactive nitrogen compounds.

In a further preferred embodiment, the apparatus is characterized in that the reactive oxygen species comprises singlet oxygen ¹O₂ and hyperoxide anions and the reactive nitrogen species comprises nitric oxide, wherein the nitric oxide reacts with the hyperoxide anions to form peroxinitrites ONOO⁻ and ONOOH.

In particular, the ROS are preferably just as stable as the singlet oxygen and interact with the target molecules and/or further radicals are formed, which are also long-lasting. Preferably, hyperoxide anions O₂·⁻ are formed in the plasma, which oxidize nitric oxide to the aforementioned RNS peroxinitrites ONOO⁻ and ONOOH.

The range of the effect can be easily calculated over the lifespan of the reactive species. For example, the average flow velocity of the air flowing through the fan can be approx. 16 m/s. Peroxinitrites have a lifespan of approx. 10 ms, such that they can still have an effect at a distance of 16 cm from the plasma generator.

The singlet oxygen, for example, has a lifespan of approx. 50 ms, such that even longer distances of approx. 80 cm from the plasma generator are possible with the singlet oxygen. Efficient disinfection of body areas, especially hands, and/or the surrounding room air is particularly possible if the air flow is enriched with a sufficiently high quantity of reactive species. The plasma generator comprising the plasma rods advantageously enables optimum enrichment with ROS and/or RNS.

In particular, ozone O₃ can also be formed in the plasma. Ozone, a gas under standard conditions, is a strong oxidizing agent that develops its oxidizing effect even at room temperature. Due to its strong oxidizing effect, the gas is already unstable at room temperature. The effect of ozone is caused by the atomic oxygen produced during the decomposition of the molecule, which is itself highly reactive and has an oxidizing effect. As a so-called “active oxygen”, ozone is therefore virtually a carrier of this reactive atomic oxygen. It is precisely the disinfecting effect of ozone on organic compounds, such as bacteria or viruses, that makes ozone treatment an important process. In contrast to chlorine as an alternative oxidizing and/or disinfecting agent, ozone treatment is above all largely environmentally friendly. The compounds decomposed by the ozone are biodegradable and even the unused ozone after the decomposition reaction decomposes independently, leaving only oxygen as a decomposition product. The cold plasma caused by the dielectric barrier discharge is generated in the plasma generator. If pure oxygen is now passed through the plasma generator, the oxygen molecules are split into atomic oxygen, which can then recombine to form ozone.

In a further preferred embodiment, the apparatus is characterized in that the air flow circulates in a circuit, the apparatus preferably comprising an activated carbon filter in the circulating air flow, which intercepts pollutants in the air flow.

The reactive species generated by the plasma generator are carried further by the air flow, in particular into a disinfection chamber, where the body areas, in particular the hands, can be held for drying and/or disinfection. There is preferably a negative pressure in the disinfection chamber so that the air flow can continue to the activated charcoal filter. The pressure ratio between the negative pressure in the disinfection chamber and the pressure in the environment is greatly advantageous, as this prevents pollutants from entering the environment. The process starts again with the purified air, creating a cycle.

For the purposes of the invention, activated charcoal filters preferably refer to filters that contain activated charcoal. A filter retains unwanted substances from the air flow, similar to a sieve. Activated charcoal preferably has a very large inner surface which adsorbs dissolved particles. The charcoal also acts as a reducing agent and can absorb oxidizing agents such as ozone and/or chlorine from the air flow. During filtering and adsorption, the substances to be removed are absorbed by the activated charcoal and enriched in the charcoal mass. During reduction, on the other hand, the charcoal is partially oxidized to carbon dioxide and thus consumed. The amount of activated charcoal is reduced and must be topped up occasionally. Filtering absorbed substances, especially solids, increases the filter resistance. The absorbed substances, especially solids, must be removed again by backwashing from the filter bed. If necessary, the filter can also be regenerated by washing, heating or replacing the activated charcoal. Substances absorbed by adsorption accumulate in the charcoal. Depending on the type of activated charcoal and the nature of the adsorbed substances, accumulations of 10 to around 20 percent by weight are possible before a perforation occurs.

Activated charcoal, also known as medical charcoal, is porous, fine-grained carbon with a large inner surface area that is often used as an adsorbent. Activated charcoal is used granulated or pressed in tablet form (charcoal tablets). The pores are open-pored and interconnected like a sponge.

In a further embodiment, the apparatus is characterized in that the fan draws in air from an environment and is released into the environment after flowing through the plasma generator.

Preferably in this embodiment, the plasma output is less than 10 W (watts), preferably between 1 W (watt) and 10 W, particularly preferably approx. 2 W or less. This reduces ozone production to a permissible level, such that an activated charcoal filter can be omitted. In this case, the air is drawn in exclusively from the surroundings by the fan. The air flow is still directed towards the plasma generator by the fan and then transferred to areas of the body, in particular hands, for drying and/or disinfection. However, the air is not circulated but released into the environment. This embodiment is particularly advantageous as more reactive species are released into the environment so that they can eliminate spores, viruses, germs and/or bacteria in the room air itself, for example in aerosols.

Both the embodiment of using an air flow circuit and the variant without the use of a circuit lead advantageously to an improvement in the room air, as the air drawn in is always decontaminated by the fan.

In a further embodiment, the invention is characterized in that the fan is equipped with one or more heating elements.

For the purposes of the invention, a heating element is a device that supplies heat to the air drawn in. Electric heating elements are the most common form of heating element. Electric heating elements convert electric current into heat. They usually contain a current-carrying heating coil that is electrically insulated from the air flow to be heated. Other heating elements known in the prior art, such as Peltier elements, can also be used depending on the application.

The heating element is used to heat the intake air. The heat causes the intake air to reach a higher temperature. The higher temperature means that areas of the body, especially hands, can be dried after washing, as the water or moisture on the hands evaporates due to the warm air.

In a further preferred embodiment, the apparatus is characterized in that the apparatus comprises a disinfection chamber into which components of the plasma, preferably comprising reactive oxygen and/or nitrogen species, are transported with the air flow, such that areas of the body, preferably hands, are disinfected from germs, bacteria and/or viruses within the disinfection chamber.

The target molecules of the reactive species are, in particular, possible pathogens that are found on the human skin of body areas, especially the hands. Possible pathogens can be the aforementioned germs, bacteria and/or viruses. Human hands touch countless objects every day, such as door handles, keyboards, kettles, books, etc., which tend to be used not just by one person, but by a large number of people. Hand disinfection is therefore particularly desirable.

Amino acids form a common basic structure of germs, bacteria and/or viruses. Amino acids are chemical compounds with a nitrogen-containing amino group and a carbon and oxygen-containing carboxylic acid group. In particular, some amino acids comprise a carbon double bond C═C. In fact, amino acids are found in all living organisms. They are the building blocks of proteins and are released when proteins are broken down.

The reactive species, in particular the singlet oxygen, reacts directly with carbon double bonds of the amino acids in the nanosecond range. These are then oxidized to peroxides, which advantageously eliminate and/or inactivate the potential pathogens via protein, DNA and/or membrane damage.

An additional supporting parallel process runs via the presence of peroxinitrites. These react with the target molecules according to the same active principle in a liquid phase. This is particularly the case if you have used a lot of water when washing your hands and your hands are very moist. The water or moisture on the hands has a disinfecting effect due to the peroxinitrites and/or the reactive oxygen and/or nitrogen species. The disinfectant therefore acts simultaneously during the process of drying the hands after washing. In particular, the disinfectant acts in addition to drying the hands. Advantageously, there is no longer any risk of scattered water droplets and/or water splashes spreading possible pathogens.

In a further preferred embodiment, the apparatus is characterized in that there is a spatial separation between the disinfection chamber and the plasma generator, wherein the apparatus is preferably designed as a two-chamber system, wherein a separation is provided between a disinfection chamber accessible to human hands and a reaction chamber comprising the plasma generator, and wherein the air stream flows through the reaction chamber before entering the disinfection chamber.

The spatial separation between the disinfection chamber and the reaction chamber is necessary as the reaction chamber must not be touched by the body areas, especially the hands. As a high alternating voltage is preferably applied to the plasma generator to create the plasma, touching it with your hands, for example, would be dangerous. The flow of electric current through a person can lead to electrical accidents, in particular to life-threatening injuries or even death. The term touch voltage is also a common term in the prior art.

The spatial separation between the disinfection chamber and the reaction chamber is preferably so large that the high alternating voltage applied no longer poses any danger to the user and at the same time the lifespan of the reactive species is sufficient to reach the body areas, in particular the hands, in order to eliminate possible pathogens. Preferably, the distance between the reaction chamber and the disinfection chamber can be at least 1 cm and preferably up to 100 cm, particularly preferably up to 80 cm. The spatial separation between the reaction chamber and the disinfection chamber advantageously solves safety-critical factors.

In a further aspect, the invention relates to a use of the apparatus according to the invention for disinfecting areas of the body, preferably hands.

The apparatus according to the invention can dry and simultaneously disinfect areas of the body, in particular hands, without contact. Additional disinfectants after hand washing and drying can thus be dispensed with. In particular, the apparatus according to the invention improves the quality of the ambient room air, as the air drawn in by the fan is decontaminated.

In a further aspect, the invention relates to a method for disinfecting areas of the body by means of the apparatus according to the invention, characterized in that the fan is used to generate an air flow which is guided through a plasma generator located in the air flow, and reactive oxygen and nitrogen species are produced in an area around the plasma generator, with which body areas, preferably hands, are disinfected.

The reactive species, which are preferably generated by the plasma generator, are transported further into the disinfection chamber by the air flow and are present there to disinfect areas of the body, in particular hands. At the same time, the body areas, in particular the hands, are dried. The water or moisture on the hands has a disinfecting effect. The reactive species, preferably the singlet oxygen, react with the carbon double bonds C═C of the amino acids. This oxidizes them to peroxides, such that the potential pathogens are eliminated and/or inactivated via protein, DNA and/or membrane damage.

Furthermore, the presence of peroxinitrites preferably introduces a further parallel process for disinfecting the body areas, preferably the hands, which reacts with the target molecules in a liquid phase according to the same active principle. This is particularly the case if the body areas, preferably the hands, are still wet after washing. Advantageously, the water or the moisture on the hands has a disinfecting effect, such that the disinfection works during drying.

The average person skilled in the art will recognize that technical features, definitions and advantages of preferred embodiments disclosed for the apparatus according to the invention apply equally to the use of the apparatus according to the invention for disinfecting body areas, preferably hands, and a method for disinfecting body areas by means of the apparatus according to the invention, and vice versa.

The invention will be explained and illustrated in more detail below by means of examples, without being limited to these.

### BRIEF DESCRIPTION OF THE DRAWINGS

FIG. 1 Illustration of a preferred embodiment of the apparatus according to the invention

FIG. 2 Illustration of a further preferred embodiment of the apparatus according to the invention

FIG. 3 Illustration of a plasma rod

FIG. 4 Illustration of the results of a test procedure according to EN 1500 with Escherichia coli K12 to prove the disinfection effect of the apparatus according to the invention

FIG. 5 Illustration of the results of a test procedure according to EN 1500 with Enterococcus faecium to prove the disinfection effect of the apparatus according to the invention

### DETAILED DESCRIPTION OF THE INVENTION

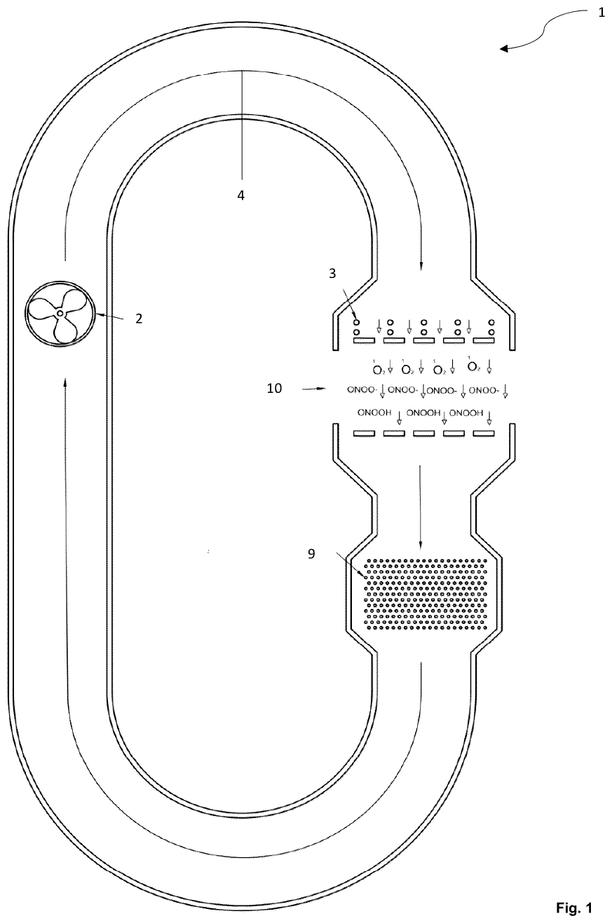

FIG. 1 is an illustration of a preferred apparatus 1 for disinfecting and/or drying areas of the body, preferably hands. The apparatus 1 comprises a fan 2, which draws in air from the environment and generates an air flow 4. Heating elements, which are not shown, heat the air drawn in. The air flow 4 runs over a flow channel. The apparatus also comprises a plasma generator 3. The plasma generator 3 is located inside a reaction chamber. The design of the plasma generator 3 as an array is shown in the illustration. A high alternating voltage is applied to the plasma generator 3. When the alternating voltage is applied, a so-called cold plasma is formed on the plasma generator 3 according to the principle of dielectric barrier discharge. Reactive species are formed in the process. Not all reactive species are long-lasting, but reactive oxygen species (ROS) and reactive nitrogen species (RNS) are an exception. The ROS are as stable as the singlet oxygen ¹O₂ and/or radicals are formed, which are also long-lasting. For example, the superoxide radical O₂·⁻, which reacts with nitric oxide to form reactive RNS comprising peroxinitrites ONOO⁻ and ONOOH, is formed in the plasma.

The singlet oxygen ¹O₂ and the peroxinitrites ONOO⁻ and ONOOH are also shown in FIG. 1. The small arrows indicate the direction in which these reactive species are transported. They enter a disinfection chamber 10 via the air flow 4, which is illustrated by the elongated curved arrow. Within the disinfection chamber, areas of the body, in particular hands, can be introduced and held for drying and/or disinfection. The target of the reactive species or the target molecules are amino acids from possible pathogens such as fungi, spores, germs, bacteria and/or viruses.

In particular, the singlet oxygen ¹O₂ reacts with the carbon double bonds of the amino acids, which is not shown in FIG. 1, and oxidizes them to peroxides, which eliminates and/or inactivates the potential pathogens through protein, DNA and/or membrane damage. There is a negative pressure in the disinfection chamber so that the air flow continues to an activated carbon filter 9. The activated carbon filter 9 traps pollutants from the air flow. The pressure ratio between the negative pressure in the disinfection chamber 10 and the pressure in the environment ensures that no pollutants enter the environment. The process starts again with the purified air, creating a cycle.

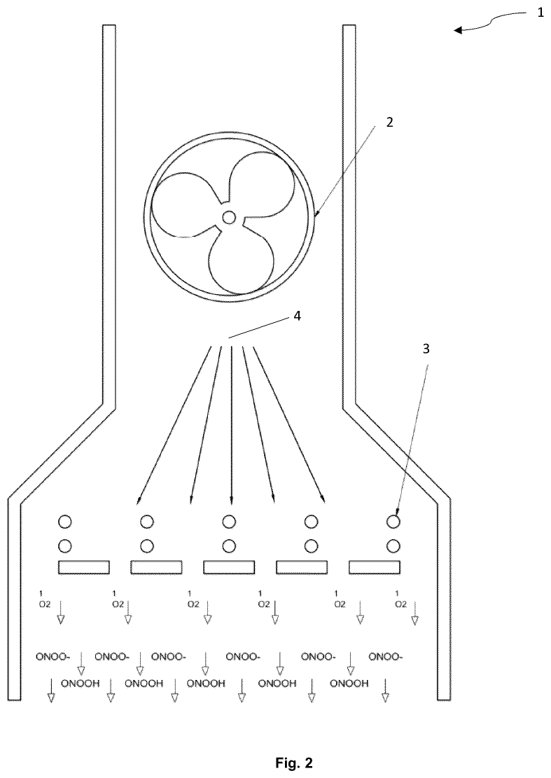

FIG. 2 is an illustration of a further preferred embodiment of the apparatus 1 for disinfecting and/or drying areas of the body, preferably hands. Preferably, this embodiment is suitable for plasma outputs of up to 2 W. Ozone production is reduced so that an activated charcoal filter can be dispensed with. In contrast to the embodiment in FIG. 1, the air is drawn in by the fan 2 exclusively from the environment and is not transferred into a circulation system, but is released back into the environment. The air drawn in is again heated by heating elements, which are not shown. The air flow 4, which is visualized by the elongated arrows, is conveyed towards the plasma generator 3 via the flow channel.

The operating principle remains identical to the embodiment shown in FIG. 1. Reactive species, in particular ROS and RNS, are formed at the plasma generator 3. The singlet oxygen ¹O₂ and the peroxinitrites ONOO⁻ and ONOOH are shown. These reactive species are transported further by the air flow 4 so that they dry and/or disinfect areas of the body and/or the ambient air. The singlet oxygen reacts with the amino acids of possible pathogens such as spores, fungi, germs, bacteria and/or viruses, which are then oxidized to peroxides so that these pathogens are illuminated via protein, DNA and/or membrane damage. The additional supporting process through the presence of peroxinitrites reacts in the same way and is particularly effective if the hands still have a high moisture content after washing.

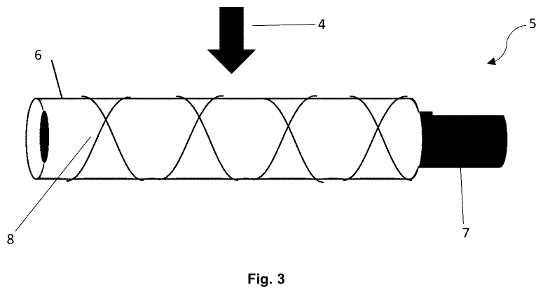

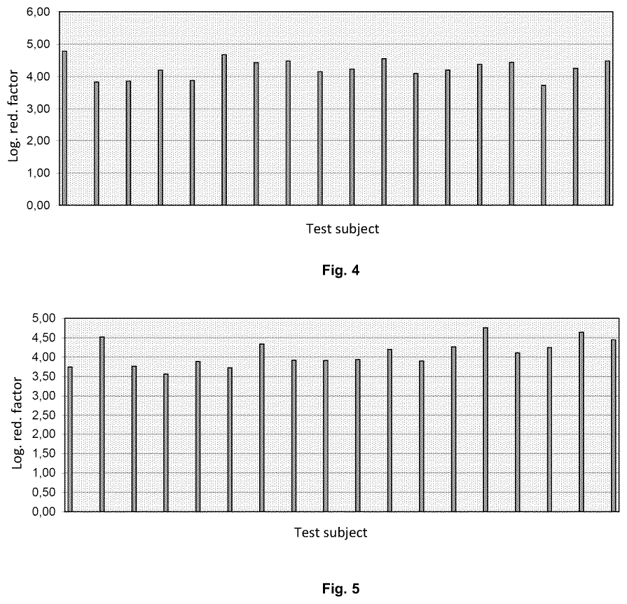

FIG. 3 shows a possible structure of a plasma rod 5, which is surrounded by the plasma generator 3. A wire 8 is coiled around a dielectric tube 5. An electrically conductive core 7 is present inside the dielectric tube 5. The wire 8 and the core 7 form a pair of electrodes. When a high alternating voltage is applied, a so-called cold plasma is formed on the outside of the dielectric tube 5 according to the principle of dielectric barrier discharge. This results in the formation of the reactive species mentioned above, which are transported further by the air flow 4, for example into the disinfection chamber 10, which is not shown in the figure. The air flow 4 flows around the plasma rod 5 in a transverse direction.

As can be seen in FIG. 3, the outer wire 8 is coiled twice and in opposite directions along the surface of the dielectric tube. This compensates for the inductance of the coil. The plasma only forms along the thin line of the wire coil. The wire 8 can be coiled with almost any coil spacing, wherein the spacing between the windings is preferably up to 10 times the outer diameter, preferably between 1 and 5 times, particularly preferably between 2 and 4 times, of the dielectric tube. Therefore, the power can be reduced to a desired level despite the high frequency and high alternating voltage value without reducing the length of the dielectric tube 6. The generation of reactive species can be distributed over a greater distance, namely the length of the dielectric tube 6, and over a larger area. This means that a large flow profile can be evenly supplied with reactive species. Since the wire is selected to be very thin with a diameter between 0.1 mm and 1 mm, preferably between 0.2 mm and 0.4 mm, the surface of the dielectric tube 6 remains smooth. The surface of the dielectric tube 6 itself has a high degree of smoothness. This causes the air flow 4 to flow smoothly around it, whereby the boundary layer in which the air adheres to the surface of the dielectric tube 6 is relatively thin. The air hardly flows in this boundary layer. The reactive species must therefore diffuse through this boundary layer. The boundary layer is disturbed at the points of the thin wire 8, which improves the mixing of the reactive species with the air flow 4.

### Examples

The invention is explained in more detail with reference to the following examples. These are not intended to limit the scope of the invention, but represent preferred embodiments of aspects of the invention which serve to better illustrate the invention described herein.

The examples show that an apparatus with a plasma generator described herein with at least one plasma rod comprising a dielectric tube on which a coiled wire is present leads to particularly good results with regard to the disinfection of body areas, in particular hands.

### Design and Operating Parameters of the Apparatus

In the tests described below for disinfecting hands, an apparatus as described above was used for disinfecting body areas, which has a fan 2 for generating an air flow 4 and a plasma generator 3, whereby the plasma generator 3 is located in the air flow 4.

The plasma generator 3 comprised two plasma rods 5, each of which had a dielectric tube 6 with an electrically conductive core 7 inside the dielectric tube 6.

The dielectric (insulating) tube 6 was made of borosilicate glass and had an outer diameter of 8 mm. The inner diameter was 5 mm.

The electrically conductive core 7 was configured in the form of a tube consisting of a wire mesh (copper) with dimensions such that the electrically conductive core 7 fits into the dielectric tube 6.

The dielectric (insulating) tube 6 had a wire 8 coiled into windings around the outside. The wire 8 was made of a copper-nickel alloy and had a diameter of 0.4 mm.

On each of the dielectric (insulating) tubes 6, the wire 8 had eight windings, which were distributed over a length of 80 mm, the wire 8 being characterized by a preferred counter-rotating coil, in which the wire was coiled around the dielectric tube 6 with four windings forwards and four windings backwards (with a reverse direction of rotation). The distance between the coils of the wire in one coiling direction was approx. 20 mm and therefore 2.5 times the outer diameter of the dielectric tube (8 mm).

In the preferred plasma generator 3, the two plasma rods 5 were connected in series. The respective electrically conductive cores 7 (or inner conductors) were connected to two output terminals of a high-voltage generator, while the coiled wires 8 were connected to each other on the outside of the dielectric (insulating) tubes 6.

The dielectric tubes 6 thus formed pairs of electrodes with the wires 8 coiled around the respective outer side, which generated a plasma when a voltage was applied.

An alternating voltage was used for the applied voltage. A high-voltage source was used as the voltage source, which allows high-voltage pulses with adjustable amplitude and repetition rate.

Preferred voltage pulses have the form of a decaying sine wave. The voltage therefore substantially consisted of a positive and a similarly sized negative half-wave, followed by a plurality of post-oscillations. In this example, a frequency of a decaying alternating voltage of approx. 180 kHz was used, although other frequencies, in particular between 1 kHz (kilohertz) and 500 kHz, can advantageously also be used.

An alternating voltage of 10 kV peak-to-peak was used for the applied voltage. The repetition rate of the voltage pulses was approx. 1.5 kHz.

The electrical output of the plasma generator at the aforementioned settings was approx. 1.5 W. The plasma source was present inside the used hand dryer directly (approx. 10 mm) in front of the air outlet.

The fan 2 for generating an air flow 4 was set for a flow rate of around 40 l/s (liters per second). The apparatus used for the tests did not have a heater. The air is blown out by the apparatus via a plurality of narrow slots. This results in a high flow velocity at the outlet. As a result, some of the moisture from the hands is blown away. Further drying of the skin takes place via the fast air flow and is supported by the fact that the air is heated as it passes through the fan 2 and due to the compression and friction in the fan 2 blower and the outlet slots.

### Example 2—Test Method According to EN 1500 with Escherichia coli K12 Using the Example of 18 Test Subjects

To prove the disinfection effect of the apparatus comprising a plasma generator according to the invention, a test procedure for hygienic hand disinfection according to EN 1500 with Escherichia coli K12 was carried out using 18 test persons as an example.

### Test Conditions:

| | |
|---|---|
| Test germ | Escherichia coli K12 |
| Exposure | 20 sec. in the air stream of the hand dryer with plasma |
| time | reaction products (according to the parameters of the |
| | previous example 1) after previous hand washing |
| Incubation | 48 h at 44° C. |
| Post-incubation | 8 h at 44° C. |
| Test standard: | EN 1500 (DIN EN 1500 Chemical disinfectants and |
| | antiseptics - Hygienic hand disinfection - Test |
| | procedure and requirements (phase 2/stage 2); German |
| | version EN 1500: 2013) |
| Culture | Colichrom-agar (coliform chromogenic agar, CCA) with |
| medium: | inhibition of the gram-positive accompanying flora and |
| | visualization of E. coli as dark blue to |
| | black colonies |

### Results:

The results of the tests on the individual test subjects are summarized in the following table:

TABLE 1
Results of a test procedure using the apparatus according to the invention in accordance
with EN 1500 with Escherichia coli K12 using the example of 18 test subjects

| | | | CFU/ | CFU/ | | CFU/test | Log | Log | Mean |
|---|---|---|---|---|---|---|---|---|---|
| No: | V1 | V2 | plate | preparation | Dilution | person | CFU/test | reduction | values |
| Pers 01 | 10.0 | 0.1 | 24 | 2400 | 100000 | 240000000 | 8.38 | 4.78 | 4.78 |
| before | mL | mL | | | | | | | |
| Pers 01 | 10.0 | 0.1 | 4 | 400 | 10 | 4000 | 3.6 | | |
| after | mL | mL | | | | | | | |
| Pers 02 | 10.0 | 0.1 | 4 | 400 | 100000 | 40000000 | 7.6 | 3.82 | 3.82 |
| before | mL | mL | | | | | | | |
| Pers 02 | 10.0 | 0.1 | 6 | 600 | 10 | 6000 | 3.78 | | |
| after | mL | mL | | | | | | | |
| Pers 03 | 10.0 | 0.1 | 5 | 500 | 100000 | 50000000 | 7.7 | 3.85 | 3.85 |
| before | mL | mL | | | | | | | |
| Pers 03 | 10.0 | 0.1 | 7 | 700 | 10 | 7000 | 3.85 | | |
| after | mL | mL | | | | | | | |
| Pers 04 | 10.0 | 0.1 | 14 | 1400 | 100000 | 140000000 | 8.15 | 4.19 | 4.19 |
| before | mL | mL | | | | | | | |
| Pers 04 | 10.0 | 0.1 | 9 | 900 | 10 | 9000 | 3.95 | | |
| after | mL | mL | | | | | | | |
| Pers 05 | 10.0 | 0.1 | 6 | 600 | 100000 | 60000000 | 7.78 | 3.88 | 3.88 |
| before | mL | mL | | | | | | | |
| Pers 05 | 10.0 | 0.1 | 8 | 800 | 10 | 8000 | 3.9 | | |
| after | mL | mL | | | | | | | |
| Pers 06 | 10.0 | 0.1 | 33 | 3300 | 100000 | 330000000 | 8.52 | 4.67 | 4.67 |
| before | mL | mL | | | | | | | |
| Pers 06 | 10.0 | 0.1 | 7 | 700 | 10 | 7000 | 3.85 | | |
| after | mL | mL | | | | | | | |
| Pers 07 | 10.0 | 0.1 | 48 | 4800 | 100000 | 480000000 | 8.68 | 4.43 | 4.43 |
| before | mL | mL | | | | | | | |
| Pers 07 | 10.0 | 0.1 | 18 | 1800 | 10 | 18000 | 4.26 | | |
| after | mL | mL | | | | | | | |
| Pers 08 | 10.0 | 0.1 | 15 | 1500 | 100000 | 150000000 | 8.18 | 4.48 | 4.48 |
| before | mL | mL | | | | | | | |
| Pers 08 | 10.0 | 0.1 | 5 | 500 | 10 | 5000 | 3.7 | | |
| after | mL | mL | | | | | | | |
| Pers 09 | 10.0 | 0.1 | 7 | 700 | 100000 | 70000000 | 7.85 | 4.15 | 4.15 |
| before | mL | mL | | | | | | | |
| Pers 09 | 10.0 | 0.1 | 5 | 500 | 10 | 5000 | 3.7 | | |
| after | mL | mL | | | | | | | |
| Pers 10 | 10.0 | 0.1 | 37 | 3700 | 100000 | 370000000 | 8.57 | 4.23 | 4.23 |
| before | mL | mL | | | | | | | |
| Pers 10 | 10.0 | 0.1 | 22 | 2200 | 10 | 22000 | 4.34 | | |
| Pers 11 | 10.0 | 0.1 | 28 | 2800 | 100000 | 280000000 | 8.45 | 4.54 | 4.54 |
| Pers 11 | 10.0 | 0.1 | 8 | 800 | 10 | 8000 | 3.9 | | |
| Pers 12 | 10.0 | 0.1 | 16 | 1600 | 100000 | 160000000 | 8.2 | 4.09 | 4.09 |
| Pers 12 | 10.0 | 0.1 | 13 | 1300 | 10 | 13000 | 4.11 | | |
| Pers 13 | 10.0 | 0.1 | 11 | 1100 | 100000 | 110000000 | 8.04 | 4.2 | 4.2 |
| Pers 13 | 10.0 | 0.1 | 7 | 700 | 10 | 7000 | 3.85 | | |
| Pers 14 | 10.0 | 0.1 | 19 | 1900 | 100000 | 190000000 | 8.28 | 4.38 | 4.38 |
| Pers 14 | 10.0 | 0.1 | 8 | 800 | 10 | 8000 | 3.9 | | |
| Pers 15 | 10.0 | 0.1 | 19 | 1900 | 100000 | 190000000 | 8.28 | 4.43 | 4.43 |
| Pers 15 | 10.0 | 0.1 | 7 | 700 | 10 | 7000 | 3.85 | | |
| Pers 16 | 10.0 | 0.1 | 9 | 900 | 100000 | 90000000 | 7.95 | 3.72 | 3.72 |
| Pers 16 | 10.0 | 0.1 | 17 | 1700 | 10 | 17000 | 4.23 | | |
| Pers 17 | 10.0 | 0.1 | 23 | 2300 | 100000 | 230000000 | 8.36 | 4.25 | 4.25 |
| before | mL | mL | | | | | | | |
| Pers 17 | 10.0 | 0.1 | 13 | 1300 | 10 | 13000 | 4.11 | | |
| Pers 18 | 10.0 | 0.1 | 27 | 2700 | 100000 | 270000000 | 8.43 | 4.48 | 4.48 |
| Pers 18 | 10.0 | 0.1 | 9 | 900 | 10 | 9000 | 3.95 | | |
| | | | | | | | | Mean | 4.25 |
| | | | | | | | | value log | |
| | | | | | | | | RF: | |
| | | | | | | | | Median | 4.24 |
| | | | | | | | | log RF: | |

Comparison control with 2-propanol W = 70% v/v,
according to RKI recommendation

| | | | | | | | | | |
|---|---|---|---|---|---|---|---|---|---|
| Pers 19 | 10.0 | 0.1 | 7 | 700 | 100000 | 70000000 | 7.85 | | |
| Pers 19 | 10.0 | 0.1 | 11 | 1100 | 10 | 11000 | 4.04 | 3.8 | 3.8 |

Explanations of the entries:
No = number of the test procedure (01-18 test persons regarding Hand Sanitizer HS 100; 19^{th} test person as control of the test procedure using an approved preparation)
V1 = total volume of preparation for washing off the hands
V2 = volume of the preparation from V1 that was applied to the plate
CFU = colony-forming units = germ count cultivated on the plate = minimum number of microorganisms that can be cultivated
CFU/plate = colonies counted on the plate
CFU/preparation = colony count contained in V1
Dilution = dilution factor of the dilution series under which the plate could be counted (2 to 100 CFU)
CFU/test specimen = Calculated germ count on the examined hands, taking into account CFU/plate and CFU/preparation and dilution
Log CFU/hand = decadic logarithm of the colony count by CFU/hand
Mean value log CFU/hand = mean value from the 18 test runs
Log reduction factor = Calculated reduction factor between reference sample (left hand, not disinfected) and disinfection sample (right hand, HS 100 was applied)
Median log CFU/hand = focus of the value distribution of the log. reduction factors of subjects 01 to 18

The results of the logarithmic reduction factor are also shown in FIG. 5.

### Discussion and Evaluation:

The German Society for Hygiene and Microbiology DGHM, as well as other professional associations and normative bodies (e.g. Association for Applied Hygiene, VAH) define “disinfection” as an antiseptic measure, i.e. a measure that eliminates infectiousness through microbiocidal action and thus leads to an aseptic state, i.e. a state without increased infection potential. By reducing the germ count, a final germ count is achieved after disinfection that is so low that the disinfected objects, e.g. hands, no longer pose a risk of infection.

Disinfection is achieved by reducing a high initial microbial load by a certain reduction factor. The reduction must take place to such an extent that the few remaining microorganisms no longer pose a risk of infection.

The analysis presented here proves the high effectiveness of a disinfection process using an apparatus according to the invention for drying areas of the body (here: hands) by means of an air flow with disinfecting plasma products (reactive species).

In accordance with EN 1500, a practical application test was carried out with 18 test persons against an already approved method (comparative test: 2-propanol 70% v/v according to the Robert Koch Institute RKI). In this example, Escherichia coli K12 was used as the test germ. The exposure of the hands contaminated by E. coli in the air flow of the dryer was 20 seconds.

After contamination with the test germ, the hands were washed with a non-microbiocidal soap and then dried for 20 seconds with air from the dryer.

After application of the method, the bacterial count of the hand exposed to the method to be tested (plasma disinfection) (right hand) is compared with the bacterial count without intervention (left hand, this was contaminated with E. coli, but not with the disinfection method using an apparatus according to the invention).

Germ reduction rates >3 log levels are recommended as sufficient reduction factors, i.e. germ reduction >1000-fold, or >99.9% of microorganisms are eliminated. In this case, a sufficient reduction of the microbiological load is achieved such that the requirements for disinfection in the above sense are met.

In the present hand disinfection test according to EN 1500 using an apparatus according to the invention, a reduction performance of 4.25 or 4.24 log levels was determined.

The scatter of the reduction factors in the individual test subjects is very low for microbiological studies; the median corresponds almost to the mean value of the red. factors.

This means that the current settings of the method lead to a germ reduction of more than 3 log levels (powers of ten) and thus more than sufficient disinfection in the sense of asepsis is achieved in accordance with EN 1500.

The microorganism Escherichia coli K12 used as the test germ is a test germ defined by EN 1500. The test germ is to be seen as an indicator or “proxy”.

A germ inactivation corresponding to the disinfection (>3 log levels) indicates that the majority of the relevant pathogens are also eliminated. The effectiveness of the method against E. coli indicates that the process is effective in Robert Koch Institute (RKI) efficacy class A, with the exception of mycobacteria; and in efficacy class B*.

The efficacy class B* describes a disinfection efficacy against enveloped viruses (e.g. hepatitis B and C, HI virus, measles virus, SARS-COV-2 virus).

The reduction performance therefore already meets the requirements of disinfection in terms of a reduction in germ count by >3 powers of ten (i.e. >1000-fold reduction in germ count).

The available results for carrying out a disinfection process using the apparatus according to the invention even show a reduction performance of approx. 15,800 times. This means that out of 15,800 microorganisms, only 1 microorganism survives. This corresponds to a >99.99% reduction in the number of microorganisms and at the same time considerably exceeds the requirements of EN 1500.

In further preliminary tests, it was found that although the procedure eliminates E. coli, the physiological flora of the skin (microbiome) is only affected to an insignificant degree.

In summary, it can be stated that a germ reduction performance of >4.25 or >4.24 powers of ten (log levels) can be achieved by means of the apparatus according to the invention with an exposure of 20 seconds in the air flow with plasma reaction products. This clearly exceeds the requirements of EN 1500. The effectiveness of the method corresponds to the effectiveness of a hand disinfectant recommended as a reference (test subject 19) while at the same time protecting the physiological site flora. Advantageously, however, a disinfection method using the apparatus according to the invention does not require alcohols or other liquid biocides.

Example 3-Test method according to EN 1500 with Enterococcus faecium using the example of 18 test subjects

To prove the disinfection effect of the apparatus comprising a plasma generator according to the invention, a test procedure for hygienic hand disinfection according to EN 1500 with Enterococcus faecium was carried out using 18 test persons as an example.

### Test Conditions:

| | |
|---|---|
| Test germ | Enterococcus faecium |
| Exposure | 20 sec. in the air stream of the hand dryer with plasma |
| time | reaction products (according to the parameters of the |
| | previous example 1) after previous hand washing |
| Incubation | 48 h at 44° C. |
| Post-incubation | 8 h at 44° C. |
| Test standard: | EN 1500 (DIN EN 1500 Chemical disinfectants and |
| | antiseptics - Hygienic hand disinfection - Test procedure |
| | and requirements (phase 2/stage 2); German version EN |
| | 1500: 2013) as a supplement to the confirmation of |
| | microbiocidal efficacy against gram-positive bacteria |
| Culture | Slanetz & Bartley - Agar as selective medium for |
| medium: | Enterococcus spp. (fecal streptococci) |

### Results:

TABLE 2
Results of a test procedure using the apparatus according to the invention in accordance
with EN 1500 with Enterococcus faecium using the example of 18 test subjects

| | | | CFU/ | CFU/ | | CFU/ | Log | Log | Mean |
|---|---|---|---|---|---|---|---|---|---|
| No: | V1 | V2 | plate | prepar | Dilution | test | CFU/te | reducti | values |
| Pers 01 | 10.0 mL | 0.1 mL | 5 | 500 | 10000 | 500000 | 7.7 | 3.74 | 3.74 |
| before | | | | | | | | | |
| Pers 01 | 10.0 mL | 0.1 mL | 9 | 900 | 10 | 9000 | 3.95 | | |
| after | | | | | | | | | |
| Pers 02 | 10.0 mL | 0.1 mL | 36 | 3600 | 10000 | 360000 | 8.56 | 4.51 | 4.51 |
| before | | | | | | | | | |
| Pers 02 | 10.0 mL | 0.1 mL | 11 | 1100 | 10 | 11000 | 4.04 | | |
| after | | | | | | | | | |
| Pers 03 | 10.0 mL | 0.1 mL | 11 | 1100 | 10000 | 110000 | 8.04 | 3.76 | 3.76 |
| before | | | | | | | | | |
| Pers 03 | 10.0 mL | 0.1 mL | 19 | 1900 | 10 | 19000 | 4.28 | | |
| after | | | | | | | | | |
| Pers 04 | 10.0 mL | 0.1 mL | 8 | 800 | 10000 | 800000 | 7.9 | 3.56 | 3.56 |
| before | | | | | | | | | |
| Pers 04 | 10.0 mL | 0.1 mL | 22 | 2200 | 10 | 22000 | 4.34 | | |
| after | | | | | | | | | |
| Pers 05 | 10.0 mL | 0.1 mL | 13 | 1300 | 10000 | 130000 | 8.11 | 3.88 | 3.88 |
| before | | | | | | | | | |
| Pers 05 | 10.0 mL | 0.1 mL | 17 | 1700 | 10 | 17000 | 4.23 | | |
| after | | | | | | | | | |
| Pers 06 | 10.0 mL | 0.1 mL | 9 | 900 | 10000 | 900000 | 7.95 | 3.72 | 3.72 |
| before | | | | | | | | | |
| Pers 06 | 10.0 mL | 0.1 mL | 17 | 1700 | 10 | 17000 | 4.23 | | |
| after | | | | | | | | | |
| Pers 07 | 10.0 mL | 0.1 mL | 24 | 2400 | 10000 | 240000 | 8.38 | 4.34 | 4.34 |
| before | | | | | | | | | |
| Pers 07 | 10.0 mL | 0.1 mL | 11 | 1100 | 10 | 11000 | 4.04 | | |
| after | | | | | | | | | |
| Pers 08 | 10.0 mL | 0.1 mL | 19 | 1900 | 10000 | 190000 | 8.28 | 3.92 | 3.92 |
| before | | | | | | | | | |
| Pers 08 | 10.0 mL | 0.1 mL | 23 | 2300 | 10 | 23000 | 4.36 | | |
| after | | | | | | | | | |
| Pers 09 | 10.0 mL | 0.1 mL | 9 | 900 | 10000 | 900000 | 7.95 | 3.91 | 3.91 |
| before | | | | | | | | | |
| Pers 09 | 10.0 mL | 0.1 mL | 11 | 1100 | 10 | 11000 | 4.04 | | |
| after | | | | | | | | | |
| Pers 10 | 10.0 mL | 0.1 mL | 12 | 1200 | 100000 | 120000000 | 8.08 | 3.93 | 3.93 |
| before | | | | | | | | | |
| Pers 10 | 10.0 mL | 0.1 mL | 14 | 1400 | 10 | 14000 | 4.15 | | |
| Pers 11 | 10.0 mL | 0.1 mL | 22 | 2200 | 10000 | 220000 | 8.34 | 4.2 | 4.2 |
| Pers 11 | 10.0 mL | 0.1 mL | 14 | 1400 | 10 | 14000 | 4.15 | | |
| Pers 12 | 10.0 mL | 0.1 mL | 19 | 1900 | 10000 | 190000 | 8.28 | 3.9 | 3.9 |
| Pers 12 | 10.0 mL | 0.1 mL | 24 | 2400 | 10 | 24000 | 4.38 | | |
| Pers 13 | 10.0 mL | 0.1 mL | 11 | 1100 | 10000 | 110000 | 8.04 | 4.26 | 4.26 |
| Pers 13 | 10.0 mL | 0.1 mL | 6 | 600 | 10 | 6000 | 3.78 | | |
| Pers 14 | 10.0 mL | 0.1 mL | 17 | 1700 | 10000 | 170000 | 8.23 | 4.75 | 4.75 |
| Pers 14 | 10.0 mL | 0.1 mL | 3 | 300 | 10 | 3000 | 3.48 | | |
| Pers 15 | 10.0 mL | 0.1 mL | 9 | 900 | 10000 | 900000 | 7.95 | 4.11 | 4.11 |
| Pers 15 | 10.0 mL | 0.1 mL | 7 | 700 | 10 | 7000 | 3.85 | | |
| Pers 16 | 10.0 mL | 0.1 mL | 14 | 1400 | 10000 | 140000 | 8.15 | 4.24 | 4.24 |
| Pers 16 | 10.0 mL | 0.1 mL | 8 | 800 | 10 | 8000 | 3.9 | | |
| Pers 17 | 10.0 mL | 0.1 mL | 13 | 1300 | 10000 | 130000 | 8.11 | 4.64 | 4.64 |
| Pers 17 | 10.0 mL | 0.1 mL | 3 | 300 | 10 | 3000 | 3.48 | | |
| Pers 18 | 10.0 mL | 0.1 mL | 42 | 4200 | 10000 | 420000 | 8.62 | 4.45 | 4.45 |
| Pers 18 | 10.0 mL | 0.1 mL | 15 | 1500 | 10 | 15000 | 4.18 | | |
| | | | | | | | | Mean | 4.1 |
| | | | | | | | | [symbol] | |
| | | | | | | | | Median | 4.02 |
| | | | | | | | | log | |

Comparison control with 2-propanol W = 70% v/v,
according to RKI recommendation

| | | | | | | | | | |
|---|---|---|---|---|---|---|---|---|---|
| Pers 19 | 10.0 mL | 0.1 mL | 7 | 700 | 10000 | 700000 | 7.85 | | |
| Pers 19 | 10.0 mL | 0.1 mL | 11 | 1100 | 10 | 11000 | 4.04 | 3.8 | 3.8 |

Explanations of the entries:
No = number of the test procedure (01-18 test persons regarding Hand Sanitizer HS 100; 19^{th} test person as control of the test procedure using an approved preparation)
V1 = Total volume of preparation for washing off the hands
V2 = volume of the preparation from V1 that was applied to the plate
CFU = colony-forming units = germ count cultivated on the plate = minimum number of microorganisms that can be cultivated
CFU/plate = colonies counted on the plate
CFU/preparation = colony count contained in V1
Dilution = dilution factor of the dilution series under which the plate could be counted (2 to 100 CFU)
CFU/test specimen = Calculated germ count on the examined hands, taking into account CFU/plate and CFU/batch and dilution
Log CFU/hand = decadic logarithm of the colony count by CFU/hand
Mean value log CFU/hand = mean value from the 18 test runs
Log reduction factor = Calculated reduction factor between reference sample (left hand, not disinfected) and disinfection sample (right hand, HS 100 was applied)
Median log CFU/hand = focus of the value distribution of the log. reduction factors of subjects 01 to 18 [symbol]
indicates data missing or illegible when filed

The results of the logarithmic reduction factor are also shown in FIG. 5.

### Discussion and Evaluation:

The German Society for Hygiene and Microbiology DGHM, as well as other professional associations and normative bodies (e.g. Association for Applied Hygiene, VAH) define “disinfection” as an antiseptic measure, i.e. a measure that eliminates infectiousness through microbiocidal action and thus leads to an aseptic state, i.e. a state without increased infection potential. By reducing the germ count, a final germ count is achieved after disinfection that is so low that the disinfected objects, e.g. hands, no longer pose a risk of infection.

Disinfection is achieved by reducing a high initial microbial load by a certain reduction factor. The reduction must be carried out to such an extent that the few remaining microorganisms no longer pose a risk of infection.

The analysis presented here proves the high effectiveness of a disinfection process using an apparatus according to the invention for drying areas of the body (here: hands) by means of an air stream with disinfecting plasma products.

In accordance with EN 1500, a practical application test was carried out with 18 test persons against an already approved method (comparative test: 2-propanol 70% v/v according to the Robert Koch Institute RKI). In this example, Enterococcus faecium was used as the test germ. The exposure of the hands contaminated by Enterococcus faecium in the air flow of the dryer was 20 seconds.

After contamination with the test germ, the hands were washed with a non-microbiocidal soap and then dried for 20 seconds with air from the dryer.

After application of the method, the germ count of the hand exposed to the method to be tested (plasma disinfection) (right hand) is compared with the bacterial count without intervention (left hand, which was contaminated with Enterococcus faecium but not subjected to the disinfection method using an apparatus according to the invention.

Germ reduction rates >3 log levels are recommended as sufficient reduction factors, i.e. germ reduction >1000-fold, or >99.9% of the microorganisms are eliminated. In this case, a sufficient reduction of the microbiological load is achieved such that the requirements for disinfection in the above sense are met.

In the present hand disinfection test according to EN 1500 using an apparatus according to the invention, a reduction performance of 4.10 or 4.02 log levels was determined.

The variance of the reduction factors in the individual subjects is very low for microbiological studies; the median is almost equal to the mean value of the red. factors

This means that the current settings of the process lead to a germ reduction of more than 3 log levels (powers of ten) and thus more than sufficient disinfection in the sense of asepsis is achieved in accordance with EN 1500.

The Enterococcus faecium used as the test germ in this example supplements E. coli of EN 1500 in order to investigate its effectiveness against gram-positive bacteria.

The test germ should be seen as an indicator or “proxy”. A germ inactivation corresponding to the disinfection (>3 log levels) indicates that the majority of the relevant pathogens are also eliminated. The effectiveness of the method against Enterococcus faecium indicates that the process is effective in the Robert Koch Institute (RKI) efficacy class A, with the exception of mycobacteria; and in efficacy class B*.

The efficacy class B* describes a disinfection efficacy against enveloped viruses (e.g. hepatitis B and C, HI virus, measles virus, SARS-COV-2 virus).

The reduction performance therefore already meets the requirements of disinfection in terms of a reduction in bacterial count by >3 powers of ten (i.e. >1000-fold reduction in bacterial count).

The available results for carrying out a disinfection method using the apparatus according to the invention even show a reduction performance of approximately 12,500 times. This means that out of 12,500 microorganisms, only 1 microorganism survives. This corresponds to a >99.99% reduction in the number of microorganisms and at the same time considerably exceeds the requirements of EN 1500.

In further preliminary tests, it was found that the procedure eliminates Enterococcus faecium, but only affects the physiological local flora of the skin to an insignificant degree (microbiome).

In summary, it can be stated that a germ reduction performance of >4.10 or >4.02 powers of ten (log levels) for Enterococcus faecium can be achieved by means of the apparatus according to the invention with an exposure of 20 seconds in the air flow with plasma reaction products. This clearly exceeds the requirements of EN 1500. The efficacy of the method corresponds to the efficacy of a hand disinfectant recommended as a reference (test subject 19) while at the same time protecting the physiological site flora. Advantageously, however, a disinfection process using the apparatus according to the invention does not require alcohols or other liquid biocides.

1. Apparatus for disinfecting areas of the body comprising a fan for generating an air flow and a plasma generator, wherein the plasma generator is present in the air flow and the plasma generator comprises at least one plasma rod, which comprises a dielectric tube with an electrically conductive core inside the dielectric tube, the dielectric tube exhibits on an outer side a wire coiled into windings, and wherein the electrically conductive core forms with the wire coiled around the outer side a pair of electrodes which generates a plasma when a voltage is applied.

2. Apparatus according to claim 1, wherein the plasma generator comprises an array of a plurality of plasma rods.

3. Apparatus according to claim 1, wherein the dielectric tube exhibits a wall thickness of between 0.5 mm and 3 mm.

4. Apparatus according to claim 1, wherein the dielectric tube exhibits a length of 4 to 30 times the outer diameter of the tube.

5. Apparatus according to claim 1, wherein the wire is coiled into windings in opposite directions over at least twice the length of the tube and a plasma distributed over the length of the tube is generated as a function of the applied voltage.

6. Apparatus according to claim 1, wherein the windings of the wire have a distance from one another of between 1 and 10 times the outer diameter.

7. Apparatus according to claim 1, wherein the wire exhibits a diameter of between 0.1 mm and 1 mm.

8. Apparatus according to claim 1, wherein the electrically conductive core comprises a metal wire.

9. Apparatus according to claim 1, wherein the apparatus comprises a voltage source and/or a transformer for providing a desired voltage.

10. Apparatus according to claim 1, wherein the applied voltage is an alternating voltage.

11. Apparatus according to claim 1, wherein the plasma comprises a reactive oxygen and nitrogen species.

12. Apparatus according to claim 11, wherein the reactive oxygen species comprises singlet oxygen ¹O₂ and hyperoxide anions and/or the reactive nitrogen species comprises nitrogen monoxide, wherein the nitrogen monoxide reacts with hyperoxide anions to form peroxinitrites ONOO⁻ and ONOOH.

13. Apparatus according to claim 1, wherein the air flow circulates in a circulation system.

14. Apparatus according to claim 1, wherein the fan draws in air from an environment and is released into the environment after flowing through the plasma generator.

15. Apparatus according to claim 1, wherein the fan is equipped with one or more heating elements.

16. Apparatus according to claim 1, wherein the apparatus comprises a disinfection chamber, into which components of the plasma are transported with the air flow (4), such that areas of the body; are disinfected from germs, bacteria and/or viruses within the disinfection chamber.

17. Apparatus according to claim 1, wherein there is a spatial separation between the disinfection chamber and the plasma generator, wherein the apparatus is preferably designed as a two-chamber system, wherein a separation is provided between a disinfection chamber accessible to human hands and a reaction chamber comprising the plasma generator, and wherein the air flow flows through the reaction chamber before entering the disinfection chamber.

18. Use of an apparatus according to claim 1 for disinfecting areas of the body.

19. Method for disinfecting areas of the body by means of an apparatus according to claim 1, wherein an air flow is generated with the aid of a fan, which air flow is guided through a plasma generator present in the air flow and wherein reactive oxygen and nitrogen species result in a region of the plasma generator around which the air flows, with which body areas are disinfected.
